(19) United States
(12) Patent Application Publication
(10) Pub. No.: US 2020/0364647 A1
(43) Pub. Date: Nov. 19, 2020

(54) System and Method for Monitoring Environmental Conditions of a Physical Item During Transportation

(71) Applicant: modum.io AG, Zurich (CH)

(72) Inventor: Marc Degen, Kilchberg (CH)

(21) Appl. No.: 16/464,343

(22) PCT Filed: Nov. 28, 2017
(86) PCT No.: PCT/EP2017/080701

§ 371 Date: May 28, 2019

(30) Foreign Application Priority Data
Nov. 29, 2016 (EP) 16201028.4

(51) Int. Cl. G06Q 10/08

(52) U.S. Cl. CPC G06Q 10/083; G01N 33/0037

(57) ABSTRACT

A method is disclosed for verification of environmental data relating to the conditions under which perishable goods are stored and/or transported. The method comprises measuring, by one or more sensors employed by storage facilities, a physical quantity that relates to environmental conditions to which the perishable goods are subjected to; receiving, at a plurality of computing devices, data descriptive of the measured physical quantity; checking the validity of the received data by at least one of the plurality computing devices to determine if the conditions relating to a smart contract are met; and to ensure data integrity, creating, transmitting and storing transaction data relating to the received environmental data in a plurality of distributed public ledgers, if the received environmental data is found to be valid, wherein the plurality of distributed public ledgers are part of a blockchain system and the transaction data are stored in a blockchain.

### TECHNICAL FIELD

The present disclosure relates to the monitoring of environmental conditions during transportation of physical perishable items according to claim 1 and comprises a system therefor.

### PRIOR ART

The environmental conditions within a cargo compartment used for transporting physical perishable items, products and/or goods items may have to meet certain regulatory requirements, which can encompass or define quality requirements, e.g., to ensure that the quality of the product delivered to the end-user is maintained.

These regulatory requirements may depend on the type of product to be transported and concern, for example, the temperature and humidity in the cargo compartment. Comparatively stringent regulatory requirements may for example be applied with regard to the storage and/or transportation of food, pharmaceutical, biological and/or chemical products.

The shipment of cargo may be categorized as local or non-local. Non-local shipment may involve more handling steps than the local shipment and may utilize a wider range of shipment vehicles such as trains, planes, ships and tractor trailers.

Both local and, even more so, non-local shipment, can pose significant challenges to the cargo handler who has to make sure that the environmental conditions imposed by the regulatory are met along a transportation route, and to the end-user who is interested in validating the quality of the received product.

US 2004/049428 discloses a system for monitoring an environmental condition associated with an item of inventory along a distribution chain having a plurality of locations. The system comprises at least one item of inventory, an RF transponder associated with the item of inventory, and at least one environmental condition sensor in communication with the RF transponder. The system includes a power source for powering the RF transponder and the environmental condition sensor to record an environmental condition. The system also includes a log of location data, a log of environmental condition data, and a reporting infrastructure for processing the location data and the environmental condition data. A method for tracking an environmental condition along a distribution chain is also included. Features from the preamble of claim 1 are disclosed in this prior art document.

WO 2015/171961 discloses a method and system to validate that storage and/or shipment of products or items that are sensitive to environmental conditions such as temperature, exposure to light, vibration, etc., which affect the efficacy and/or projected expiration date of the products or items, complies with environmental requirements for the stored and/or shipped products or items. The system does not monitor the product directly but relies on sensor information provided by the means of transportation to supervise the conditions imposed by the stored products.

U.S. Pat. No. 7,693,739 is directed to a system for collecting data concerning a supply chain, for performing statistical analysis on the collected data to facilitate identification of anomalies or inefficiencies in the process, and for communicating results of such statistical analysis to those responsible for the supply chain. A method for performing statistical analysis on monitored aspect of a product supply chain involves storing, in memory accessible to processor, first data reflecting first monitored aspect of a first shipment of first item occurring in the supply chain, and storing, in memory accessible to the processor, second data reflecting second monitored aspect of a second shipment of second item occurring in the supply chain. The processor is used to automatically generate report reflecting statistical analysis of the first and second data.

WO 2016/178990 refers to systems and methods for managing media, such as digital content, using blockchain technology. This relates to multiple digital currency transfers between address nodes to register a collection of rights to a digital content item to a blockchain, and perform a digital currency transfer transaction between address nodes to register the collection of rights to the blockchain. It explains rights for digital contact as digital contracts as part of smart contracts.

US 2016/098730 relates to a method for blockchain verification of goods includes obtaining, by a first computing device, a first address, followed by exporting said first address to a first code affixed to a first product. Then a first crypto-currency transaction to the first address is filed by the first computing device at a transaction register. The method includes receiving, by a second computing device, from a code scanner, the first address, scanned from the first code affixed to the first product followed by a verification step by the second computing device of the first crypto-currency transaction at the transaction register, using the first address. Then the method identifies by the second computing device and based on said verification that the first product is authentic.

US 2015/332283 discloses a healthcare transaction validation systems and methods. Healthcare transactions associated with a stakeholder are compiled into a chain of healthcare transaction blocks. The chain can be considered a chronicle of person's healthcare path through life. When a transaction is conducted, the corresponding healthcare parameters (e.g., inputs, outputs, clinical evidence, outcomes, etc.) are sent to one or more validation devices. The devices establish a validity of the transaction and generate a new block via a proof-of-work principle. Once the new block has been calculated it can be appended to the stakeholder's health care blockchain.

### SUMMARY OF THE INVENTION

The prior art teaches a method for verification of environmental data relating to the conditions under which perishable goods are stored and/or transported, comprising: measuring, by one or more sensors employed by one or more storage facilities, environmental data descriptive of a physical quantity that relates to environmental conditions to which the perishable goods are subjected to; receiving, at a plurality of computing devices, said environmental data descriptive of the measured physical quantity.

Based on this prior art it is an object of the present invention to provide an improved method allowing a simple verification of the correctness of the sensed environmental data.

Therefore, the method further comprises the steps of: checking the validity of the received environmental data by at least one of the plurality computing devices to determine if the conditions relating to a smart contract comprising measurement conditions for the environmental data are met; creating transaction data relating to the received environmental data based on the validity check; transmitting said transaction data as well as the received environmental data to a plurality of distributed public ledgers, if the received environmental data is found to be valid; and to ensure data integrity, storing said transaction data relating to the received environmental data in said a plurality of distributed public ledgers, if the received environmental data is found to be valid, wherein the plurality of distributed public ledgers are part of a blockchain system and the transaction data are stored in a blockchain.

It is an advantage of the above mentioned method to check the sensor data against a predetermined model of conditions to be fulfilled, written down in rules contained in a smart contract and to distribute the related transaction data to a plurality of different nodes and store them there. There is one computing device having a node and checking the rules of the smart contract, which can comprise a check whether the data is from a non-authorized device, a check of the non-functioning of the detection process or the sensor. A lost sensor is also a non-functioning sensor, i.e. when the computing device having a node is remote to the sensor in question and the container with the perishable goods and the associated sensor is lost, e.g. it cannot send any information via a network to said computing device waiting for the environmental data from this specific sensor.

Such a non-functioning sensor may comprise the absence of measured environmental data in a predetermined time window whereby lack of data is then the received environmental data. In other words, the failure of delivery of data replaces then the received environmental data. The related transaction data of this check are stored in the node's ledger. Copies of these transaction data are stored in further nodes in the same or other computing devices forming distributed ledgers. The content can be retrieved by everyone, therefore such distributed ledgers are called public ledgers. It is to be noted that the content of the transaction data can be a hash of the originally created environmental data, thus any authorized person having the original data can check the authenticity of these data, whereas third persons cannot derive the original data from the hash. It is also possible to write encrypted data of the environmental data as part of the transaction data. The blockchain approach is a specific sequential format of the distributed ledgers.

The step of measuring occurs along a transportation route of the perishable items, while the perishable items are transported, i.e. they are made at subsequent points in time and at different positions; both values can be part of the environmental data.

The invention also comprises a system to execute the above method and a computer program product comprising code to be executed on and by a computer to execute the method.

Such a system can have a plurality of sensors, wherein a plurality or all of these sensors comprise one of said computing devices. In other words; computing devices are integrated with the sensors, preferably together on one circuit board. Then these computing devices directly function as nodes of a blockchain and they are configured to be employed for validating and storing data records in respective ledgers. Then the system of sensors can be tantamount to the system of computing devices. Of course additional computing devices can be provided but the system can also be provided as a compact unit. Then the user of the system, where at any time, sensors to be used in the method are available, are also the guarantor of the blockchain nodes which facilities the maintenance of the entire system.

Of course, it is also possible that a plurality of computing devices comprise at least one of said sensors.

The method according to an embodiment of the invention can have a secret key associated with each sensor to digitally sign environmental data provided by said sensor with the associated private key for later identification purposes with a publically available public key.

This enables an auditing step since a client having sent the perishable goods with the system, according to the invention, can check hardware and the blockchain security in two steps. He retrieves the available public key of the provider of the system and sensor according to the invention. If this key is countersigned by a certified authority, then all data received from a sensor of the system can be traced to this public key. If this is correct, the data originates from authentic loggers, if not, then either the data does not originate from authentic loggers or was manipulated. This adds an additional layer of security but it is not mandatory for the invention.

In an embodiment the client receives the environmental data from the system. He can read e.g. hash values from the public ledgers, and if the checksum of the data matches with the hash code generated by him, then the measurements are as they were at the time of being recorded, if not, the received environmental data were changed and this has to be investigated. It is also possible that preferably encrypted environmental values are stored on the public ledgers which can then be accessed by the client having received the necessary key from the provider of the system. Then he can directly check the data with the also provided transaction data.

Additionally, reputation data descriptive of the reputation of a sensor can then also be associated with its public key, e.g., by computing device. Then subsequent validated sensor data increases the reputation value, whereas failure would decrease this value.

In an open system, in order to create an incentive to provide accurate environmental data, each sensor receives a tradable digital asset that can include micropayments in the form of a digital currency, which is especially proportional to its reputation.

While it is preferable to encompass positioning data in the set of environmental data, it is contemplated to nevertheless reduce sensor power consumption. Therefore, sensors, being free of any positioning units, can be associated with positioning sensors of an associated storage facility, so that they use the continuous power of a storage facility while reducing its own power needs to a minimum.

Example 1 concerns a method for blockchain verification of environmental data relating to the conditions under which perishable goods are stored and/or transported, comprising: measuring, by one or more sensors employed by one or more storage facilities, a physical quantity that relates to environmental conditions to which the perishable goods are subjected to; receiving, at a plurality of computing devices, data descriptive of the measured physical quantity; checking the validity of the received data by at least one of the plurality computing devices to determine if the conditions relating to a smart contract are met; and to ensure data integrity, storing transaction data relating to the received environmental data in a plurality of distributed public ledgers, if the received environmental data is found to be valid.

Example 2 includes the subject matter of example 1 and, optionally, wherein the step of measuring occurs along a transportation route of the perishable items, while the perishable items are transported.

Example 3 includes the subject matter of any of examples 1 or 2 and, optionally, wherein the one or more sensors include any one of the following: inertial sensors (e.g., comprising an accelerometers for implementing, for example, fall sensors) and/or non-inertial sensors, wherein the non-inertial sensors comprise at least one of the following: temperature sensors; humidity sensors; pressure sensors; light sensors; non-inertial sensors; gas sensors, e.g., for the detection of Volatile Organic Compounds; air flow sensors; and/or NOx sensors.

Example 4 includes the subject matter of any of examples 1 to 3 and, optionally, wherein the one or more sensors are employed to sense any one of the following physical quantities: temperature, pressure, relative humidity, and/or intensity of light.

Example 5 concerns a system for blockchain verification of environmental data relating to the conditions under which perishable goods are stored and/or transported, comprising: one or more sensors for measuring a physical quantity that relates to an environmental condition in and/or outside a storage facility used for storing and/or transporting perishable goods; a plurality of computing devices that are operative and configured to receive data descriptive of the measured physical quantity, wherein at least one of the plurality of computing devices is operative and configured to check the validity of the received data to determine if the conditions relating to a smart contract are met; and wherein at least two of the plurality of computing devices are operative to store transaction data relating to the received environmental data in a respective at least two distributed public ledgers, if the received environmental data is found to be valid.

Example 6 includes the subject matter of example 5 and, optionally, wherein the one or more sensors are mounted directly on the packaging of perishable goods such as, for example, blister packs.

Example 7 includes the subject matter of example 6 and, optionally, wherein the one or more sensors include any one of the following: temperature sensors; humidity sensors; pressure sensors; light sensors; accelerometers (e.g., fall sensors); gas sensors, e.g., for the detection of Volatile Organic Compounds; air flow sensors; and/or NOx sensors.

Example 8 includes the subject matter of example 7 and, optionally, wherein the one or more sensors are operative to sense any one of the following physical quantities: temperature, pressure, relative humidity, and/or intensity of light.

Example 9 concerns a transitory or non-transitory computer readable storage medium storing a set of instructions that are executable by at least one processor of a server to cause the server to perform a method for verification of environmental data relating to the conditions under which perishable goods are stored and/or transported the method comprising: measuring, by one or more sensors mounted on and/or comprised in one or more storage facilities, a physical quantity that relates to an environmental condition in a storage compartment of the storage facilities; receiving, at a plurality of computing devices, data descriptive of the measured physical quantity; checking the validity of the received data by at least one of the computing devices to determine if the conditions relating to a smart contract are met; and to ensure data integrity, storing transaction data relating to the received environmental data in a plurality of distributed public ledgers, if the received environmental data is found to be valid.

### BRIEF DESCRIPTION OF THE DRAWINGS

Preferred embodiments of the invention are described in the following with reference to the drawings, which are for the purpose of illustrating the present preferred embodiments of the invention and not for the purpose of limiting the same. In the drawings,

FIG. 1 is a schematic diagram of a blockchain-based system for monitoring environmental conditions under which perishable goods are transported, according to some embodiments;

FIG. 2 is a schematic diagram showing the components of sensors and computing devices of the system, according to some embodiments; and

FIG. 3 is a schematic flow chart diagram of a method for monitoring the environmental conditions under which perishable goods are transported, according to some embodiments.

### DESCRIPTION OF PREFERRED EMBODIMENTS

Aspects of embodiments disclosed herein relate to systems and methods allowing an end-user to use blockchain-based validation to determine if predetermined conditions laid out in a smart contract and relating to the environmental conditions during the transportation of perishable goods are met or not. The systems and methods disclosed herein thus facilitates assessing if transportation was regularly compliant and, optionally, the quality of the goods received by the end-user.

The following description of systems and methods for monitoring environmental parameters during transportation of physical items is given with reference to particular examples, with the understanding that such systems and methods are not limited to these examples.

It is noted that the terms “physical items”, “goods”, “commodities” and “products” may herein be used interchangeably. The term “physical item” as used herein may refer to any physically tangible, individually distinguishable unit of packaged or unpackaged object or objects.

The term “perishable”, “perishable”, “perishable”, and/or the like as used herein in conjunction with items, goods and/or commodities is to indicate non-durable goods that must be used within a certain period of time and which deteriorate quickly when not stored properly. Accordingly, perishable goods can be subject to decay and/or spoilage, and therefore have a limited lifetime and require proper storage. Optionally, they may require rapid transport. Perishable goods can also include hazardous goods.

Generally, a system for monitoring environmental parameters can include a plurality of sensors that are operative and configured to measure (also: sense) physical quantities relating to one or more environmental parameters pertaining to the storage compartments of storage facilities (including, for example, cargo compartments of transportation vehicles). The plurality of sensors may be communicably coupled with each other in a global network enabling peer-to-peer exchange of data including, for instance, a mesh network.

The environmental data, which is descriptive of environmental information, is generated by the plurality of sensors and stored in a blockchain. Once validated, the stored environmental data may be immutable. Hence, the valid and correct reporting of the environmental data pertaining to the storage of the perishable item to the end-user of the item is herewith ensured, and allows determining if the conditions laid out in a smart contract that defines requirements concerning regulatory compliance are met or not. Only authorized end-users may be granted access to the data in the blockchain, e.g., responsive to entering a correct response in case the data is protected by challenge-response authentication.

It is noted that regulatory requirements can encompass or refer, for example, quality requirements (e.g., of food products), transportation requirements, and/or storage requirements.

In other words, the system enables the implementation of a computerized transaction protocol that executes the terms of a smart contract relating to the monitoring of environmental data under which perishable items are stored (including, e.g., the monitoring of environmental conditions under which perishable items are transported along the transportation route) by enabling determining, for example, if the environmental conditions under which the perishable items are or were stored or transported meet the regulatory requirements as defined by the smart contract.

It is noted that while embodiments disclosed herein may specifically refer to the transportation of perishable goods, this should by no means be construed limiting. Embodiments disclosed herein can, wherever applicable, additionally or alternatively refer to the stationary storage of perishable goods.

In some embodiments, a “smart contract” may be specific for each supplier-receiver relationship. Each packaged good may be uniquely identified with an ID using, for example, a machine-readable optical label (e.g., barcode, a QR code); an RFID code; and/or an ID based on 1-wire technology, allowing the association of goods with data logged by the sensors for the implementation of a smart contract.

Data descriptive of the sensed environmental parameters are provided to an “environmental conditions monitoring (ECM) register” that comprises a plurality of distributed computing devices (also: nodes), storing a respective plurality of synchronized ledgers with validated environmental data records. These ledgers may be publicly accessible to anyone with access rights. The term “distributed” as used herein with respect to computing devices is to indicate that the computing are located remotely from each other, i.e., they are not co-located.

The plurality of distributed computing devices may comprise, for example, servers (e.g., for implementing a file hosting service and/or a cloud storage service), and/or computerized client devices. A computerized client device can include, for example, a multifunctional mobile communication device also known as “smartphone”, a personal computer, a laptop computer, a tablet computer, a personal digital assistant, a wearable device, a handheld computer, a notebook computer, a vehicular device and/or any movable object which is operative to send and/or receive data wirelessly and to process received data. The terms “computerized client device” and “client device” may herein be used interchangeably.

Environmental data received by a given computing device are vetted for their integrity, e.g., by at least by one of the plurality of computing devices of the system. This vetting process may herein also be referred to as a “data validation”, or similar. The validation may for example be performed by the given computing device that receives the environmental data and/or by other computing devices of the plurality of computing devices.

In an embodiment, the validation yields that the received environmental data confirms the integrity of the received environmental data, the data is recorded in the ledger of the corresponding “validating” computing device as a block. The said block is linked to a previous record (block) of environmental data. The ledger is thus updated to include an additional block of validated environmental data to create an updated blockchain. Due to the distributed and synchronized nature of the blockchain, the validated data remains traceable.

A ledger may thus constitute a blockchain of validated environmental data that is accessible to anyone with access rights. Once a block in a ledger of a given computing device is validated, the ledgers in the other computing devices of the ECM register are synchronized, e.g., in real-time, according to the records of the updated ledger of the given computing device.

Based on the validated environmental data (or transaction data relating to validated environmental data), it may be determined if the perishable goods were transported under environmental conditions that meet regulatory. In some embodiments, the process of determining if perishable goods were stored and/or transported under environmental conditions that meet regulatory requirements, may be accomplished during the storage and/or transportation of the goods. For instance, it may be determined in real-time if perishable goods are transported in manner which is compliant to transport regulations.

The publicly accessible ledgers of the ECM register are repeatedly synchronized and distributed, making it difficult or nearly impossible to generate and/or introduce fraudulent environmental sensor data.

The creation and linking of a block to a previous block may herein referred to as “mining”. The said “mining” or linking of data records to a previous block in the chain may only be possible if the records of the previous block were also checked and found valid.

The creation, “mining” or linking of a block to a previous block may occur, for example, in accordance with a protocol that is accepted by all “participants” or computing devices of the ECM register. As an example, the protocol may require a new block to contain a cryptographic hash describing its contents. The cryptographic hash may be required to satisfy a mathematical condition, achieved by having the new block to contain a number, called a nonce, whose value is determined after the fact by the discovery of the hash that satisfies the mathematical conditions. The protocol may be able to adjust the mathematical conditions that the discovery of the hash describing a block and satisfying the mathematical condition requires more or less steps, depending on the previous hashing attempt. Additional or alternative (blockchain or mining) protocols may be employed.

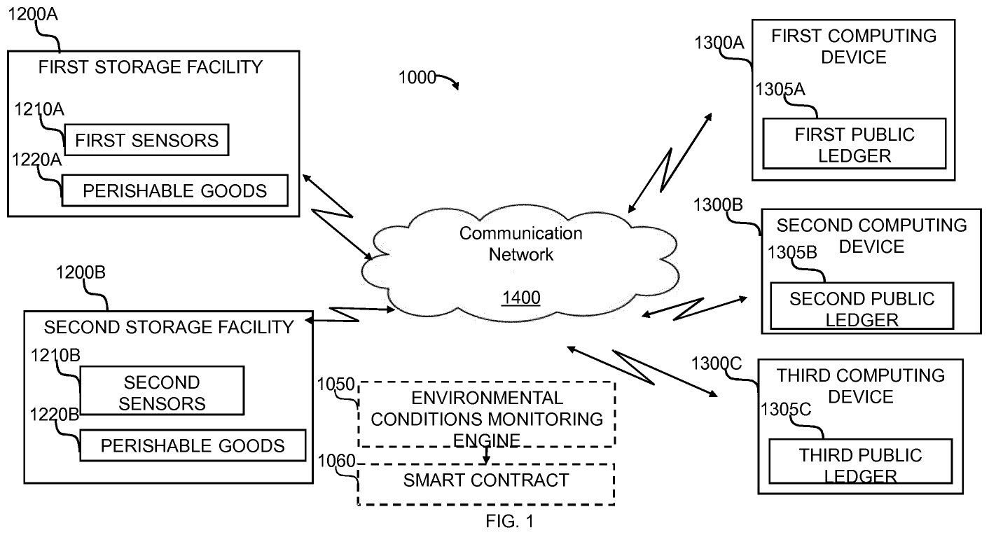

Reference is made to FIG. 1. According to some embodiments, system 1000 is operative to enable the implementation of methods, processes and/or operations for implementing a “smart contract” relating to the monitoring of environmental data along a transportation route of perishable items using blockchain technology. Such methods, processes and/or operations may herein be implemented by an “environmental conditions monitoring engine” or “ECM engine”, referenced by alphanumeric label “1050”, and the “Smart Contract” is herein referenced by alphanumeric label “1060”. The term “engine” as used herein may comprise one or more computer modules, wherein a module may be a self-contained hardware and/or software component that interfaces with a larger system. A module may comprise a machine or machines executable instructions. A module may be embodied by a circuit or a controller programmed to cause the system to implement the method, process and/or operation as disclosed herein. For example, a module may be implemented as a hardware circuit comprising, e.g., custom VLSI circuits or gate arrays, off-the-shelf semiconductors such as logic chips, transistors, and/or other discrete components. A module may also be implemented in programmable hardware devices such as field programmable gate arrays, programmable array logic, programmable logic devices and/or the like.

According to some embodiments, system 1000 is operative and configured to monitor environmental parameters relating to physical perishable goods 1220 that are stored in a storage facility 1200. In an embodiment, system 1000 comprises sensors 1210 that are operative and configured to sense one or more physical quantities relating to environmental parameters inside and/or inside storage facility 1200 to which the perishable goods can be subjected to.

Such storage facility 1200 can include, for example, a movable storage facility such as a cargo compartment mounted on a transportation vehicle (not shown). For instance, the environmental conditions pertaining to perishable goods 1220 can be monitored (e.g., continuously or substantially continuously) during their transportation along a transportation route.

A first storage facility 1200A may comprise one or more first sensors 1210A, and a second storage facility 1200B may comprise one or more second sensors 1210B. It should be noted that an identical or different numbers of sensors may be employed by various storage facilities 1200. Moreover, identical or different types of sensors may be employed by various storage facilities 1200.

In order to distinguish between each of the objects, capital alphabetic characters are added after the numerals, for example, sensors 1210A and 1210B. However, when there is no need to particularly distinguish each of the objects, they are simply and collectively referred to as objects without the capital characters after the numerals.

Non-limiting examples of sensors that may be employed include temperature sensors; humidity sensors; pressure sensors; light sensors; inertial sensors such as accelerometers (e.g., fall sensors); non-inertial sensors; gas sensors, e.g., for the detection of Volatile Organic Compounds; air flow sensors; NOx sensors; and/or the like. It should be noted that the term “sensor” may encompass a “detector”. The sensor(s) may be employed to measure, for example, air quality, temperature, pressure, relative humidity, intensity of light, and/or the like, in a movable cargo compartment along a transportation route of perishable goods 1220 and/or while being stored in a stationary storage. It is noted that environmental data may also relate to the location of a sensor. Location data may be continuously logged by and, subject to verifying that the location is correct, stored in computing devices 1300 of system 1000 as records of a ledger 1305. The environmental information can include a confidence interval or reliability metric. Computing devices 1300 may herein be collectively referred to as “ECM register”.

In some embodiments, sensors 1210 may comprise or embody computing devices and function as nodes of a blockchain that can be employed for validating and storing data records in respective ledgers. Moreover, in some embodiments, computing devices 1300 may comprise or embody environmental sensors.

Non-limiting examples of perishable goods 1220 can include medicine and food products.

In an embodiment, at least one of sensors 1210 may be mounted outside the storage facility, and at least one of sensors 1210 may be mounted inside the storage facility. In an embodiment, at least one of sensors 1210 may be mounted directly on the packing of perishable goods 1220. In an embodiment, at least one of sensors 1210 may be attached to blister packs. Optionally, a plurality of sensors may be employed that are operative to monitor the environmental condition for a respective plurality of pills or tablets.

Exemplarily, sensors 1210 may generate, responsive to being subjected to a physical stimulus, a measurable signal that represents a value relating to an environmental parameter pertaining to the corresponding storage facility 1200. For instance, first sensor(s) 1210A may generate a measurable signal that represents a value relating to an environmental parameter pertaining to first storage facility 1200A.

Data descriptive of the measurable signal may be provided, via a communication network 1400, from the sensors 1210 to one or more computing devices 1300 of system 1000.

A sensor data vetting or validation procedure may be employed by at least two of the plurality of computing device 1300 according to a protocol, which is herein referred to as environmental conditions monitoring (ECM) engine 1050.

If for example the outcome of the sensor validation procedure employed by second computing device 1300B yields an output indicating that the received sensor data is invalid, it is not stored in second public ledger 1305B. Conversely, if the outcome of the validation procedure yields an output indicating that the received sensor data is valid, it is stored in corresponding second public ledger 1305B. An update of a public ledger (e.g., second public ledger 1305B) may then be broadcast over network 1400 to the other storage devices (e.g., first computing device 1300A and third computing device 1300C) which then synchronize the records of their own public ledgers (e.g., first and third public ledger 1305A and 1305C) in accordance with the one of the updated second public ledger 1305B. It is noted that the validation procedure may be invoked by any one of the computing devices according to ECM monitoring engine 1050. Optionally, the records of the other ledgers of the respective other computing devices may be synchronized in accordance with the records of the ledger of a given computing device that completed the validation procedure first. Optionally, the sensor data provided to the computing devices may constitute a tradeable digital token having a fixed supply.

In some embodiments, a public key may be associated with environmental data that is provided by sensors 1210. The public key may be used to uniquely associate the environmental data with the sensor that produced the data for identification purposes. Reputation data descriptive of the reputation of a sensor 1210 may be associated with its public key, e.g., by computing devices 1300. Reputation of a sensor may be enhanced the more accurate the environmental data that the sensor produces. Correspondingly, reputation of a sensor may be decreased the less accurate the environmental data that the data same sensor produces. For example, a sensor may be broken or compromised and, therefore, generate inaccurate or false environmental data. To create an incentive to provide accurate environmental data, each sensor 1210 may receive a tradable digital asset that can include, for example, micropayments in the form of bitcoins and/or any other digital currency. Exemplarily, the amount of bitcoins a sensor 1210 receives may be proportional to its reputation. For instance, a sensor receives more bitcoins the better its reputation, improving its status. If a sensor provides environmental data that is identified as being comparatively inaccurate, it loses status.

ECM monitoring engine 1050 may then determine if the environmental conditions under which the perishable goods are stored (and/or transported) meet regulatory requirements, which are defined by smart contract 1060.

To reduce or minimize sensor power consumption, sensors 1210 may be free of any positioning units (e.g., GPS sensors). Optionally, positioning data that may be produced by a positioning sensor (not shown) of a storage facility 1200 that embodies a cargo compartment and/or transportation vehicle, may be associated with environmental data provided by sensors 1210. For instance, positioning data produced by a GPS sensor of second transportation vehicle 1200B may be associated with environmental data produced by second sensor(s) 1210B. Positioning data produced by a positioning sensor may be descriptive of position information including, for example, an absolute position, a relative position, a quantitative position (e.g., geographical coordinates) and/or of a qualitative position. The position information can include a distance (e.g., from an external device or from a base point). The position can include a region or zone. For example, the position can include a geographical area associated with a cell associated with a cell tower. The position can include multiple levels of granularity (e.g., a city, a zip code, a street, or geographic coordinates). The position information can include a confidence interval or reliability metric.

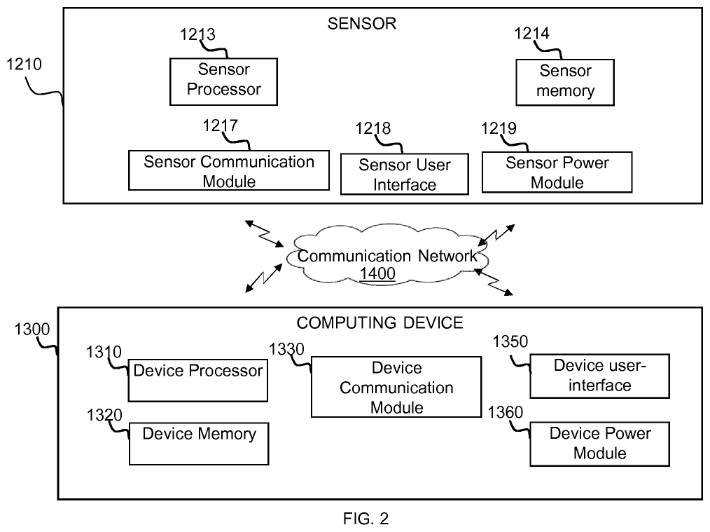

Further reference is made to FIG. 2. Sensors 1210 may in some embodiments include a sensor processor 1213, a sensor memory 1214, a sensor communication module 1217, a sensor user interface 1218, and a sensor power module 1219 for powering the various components and/or application engines and/or modules of the respective sensor 1210.

In the discussion outlined herein, components of sensors 1210A-1210B may be respectively referenced. For example, sensor processors 1213A-1213B may be included in sensors 1210A-1210B.

Computing devices 1300 may include a device processor 1310, a device memory 1320, a device communication module 1330, a device user-interface 1350, and a device power module 1360 for powering the various components of computing device 1300. In the discussion outlined herein, components of computing devices 1300A-1300B may be respectively referenced. For example, device processors 1310A-1310C may be included in computing devices 1300A-1300C.

Device user-interface 1350 may for example include a keyboard, a touchscreen, and/or the like. For example, device user-interface 1350 may be used for providing a report to the end-user, after the end-user entered a correct response via the device user-device 1350 in an embodiment where the data is protected by challenge-response authentication.

The various components of sensors 1210 and computing devices 1300 may communicate with each other over one or more communication buses (not shown) and/or signal lines (not shown).

According to some embodiments, sensor memory 1214 and computing device memory 1320 may include one or more types of computer-readable storage media including, for example, transactional memory and/or long-term storage memory facilities and may function as file storage, document storage, program storage, or as a working memory. The latter may for example be in the form of a static random access memory (SRAM), dynamic random access memory (DRAM), read-only memory (ROM), cache and/or flash memory. As working memory, sensor memory 1214 and device memory 1320 may include, for example, temporally-based and/or non-temporally based instructions. As long-term memory, sensor memory 1214 and device memory 1320 may for example include a volatile or non-volatile computer storage medium, a hard disk drive, a solid state drive, a magnetic storage medium, a flash memory and/or other storage facility. Device memory 1320 may for example store ledger 1305. A hardware memory facility may for example store a fixed information set (e.g., software code) including, but not limited to, a file, program, application, source code, object code, data, and/or the like.

The term “processor”, as used herein, may additionally or alternatively refer to a controller. A processor such as for example sensor processor 1213 and/or device processor 1310 may be implemented by various types of processor devices and/or processor architectures including, for example, embedded processors, communication processors, graphics processing unit (GPU)-accelerated computing, soft-core processors and/or general purpose processors.

Sensor communication module 1217, and device communication module 1330 may, for example, include I/O device drivers (not shown) and network interface drivers (not shown) for enabling the transmission and/or reception of data over network 1400. A device driver may for example, interface with a keypad or to a USB port. A network interface driver may for example execute protocols for the Internet, or an Intranet, Wide Area Network (WAN), Local Area Network (LAN) employing, e.g., Wireless Local Area Network (WLAN)), Metropolitan Area Network (MAN), Personal Area Network (PAN), extranet, 2G, 3G, 3.5G, 4G including for example Mobile WIMAX or Long Term Evolution (LTE) advanced, Bluetooth® (e.g., Bluetooth smart), ZigBee™, near-field communication (NFC) and/or any other current or future communication network, standard, and/or system.

Sensor memory 1214 and/or device memory 1320 may include instruction which, when executed e.g. by the respective sensor processor 1213 and/or device processor 1310, may cause the execution of the method, process and/or operation for implementing a smart contract relating to the monitoring of environmental data along a transportation route of perishable items using blockchain technology. In other words, sensor memory 1214 and/or device memory 1320 may include instruction which, when executed e.g. by the respective sensor processor 1213 and/or device processor 1310, may cause the implementation of ECM engine 1050.

Implementations and/or portions and/or processes and/or elements and/or functions of ECM engine 1050 may be implemented by sensor(s) 1210 and/or computing devices 1300.

To simplify the discussion that follows, methods and processes disclosed herein may be outlined herein in conjunction with ECM engine 1050. ECM engine 1050 may be realized by one or more hardware, software and/or hybrid hardware/software modules, e.g., as outlined herein.

ECM engine 1050 may configure system 1000 so that environmental data is provided in a distributed manner to the plurality of computing devices 1300.

It is noted that the sensors 1210 and computing devices 1300 may have to be registered in system 1000 in order to be able to take part in the blockchain.

The term “registered” and “registration” as used herein may relate to becoming part or a node of the peer-to-peer network.

Registration to system 1000 may be performed in a substantially decentralized manner, i.e., not with a central server or server system but, for example, primarily or only in communication with the end-user device itself through a data storage engine.

Optionally, registration may include (e.g., require) the installation of a software product relating to ECM engine 1050 in order to become part of the blockchain.

It is noted that the expressions “concurrently” and “in real-time” as used herein may also encompass the meaning of the expression “substantially concurrently” and “substantially in real-time”.

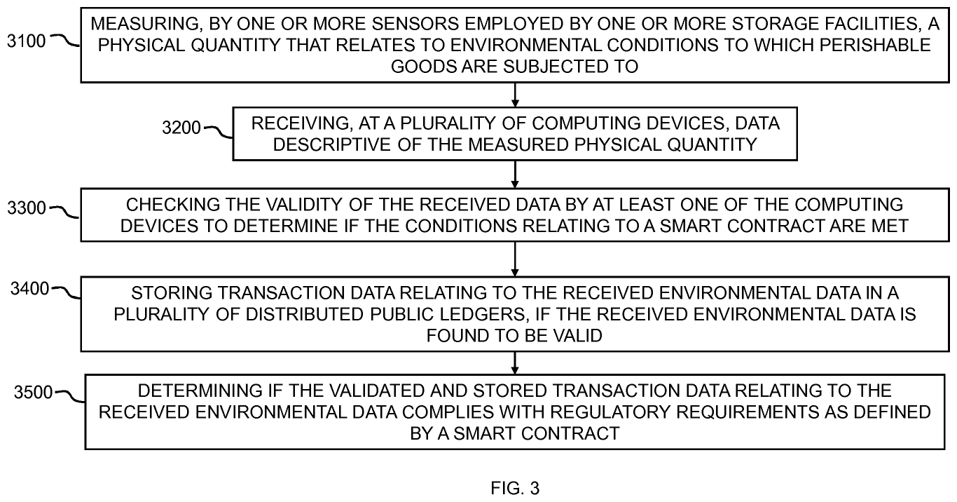

Additional reference is made to FIG. 3. As indicated by step 3100, a method for blockchain verification of environmental data relating to the conditions under which perishable goods are transported, comprises in an embodiment the step of measuring, by one or more sensors employed by one or more storage facilities, a physical quantity that relates to an environmental condition to which the perishable goods can be subjected to. For instance, the physical quantity can relate to environmental conditions inside and/or outside of the one or more storage facilities.

As indicated by step 3200, the method further includes receiving, at a plurality of computing devices, data descriptive of the measured physical quantity.

As indicated by step 3300, the method includes checking the validity of the received data by at least one of the computing devices to determine if the conditions relating to a smart contract are met.

As indicated by step 3400, the method further includes storing transaction data relating to the received environmental data in a plurality of distributed public ledgers, if the received environmental data is found to be valid.

As indicated by step 3500, the method includes determining if the validated and stored transaction data relating to the received environmental data complies with regulatory requirements as defined by smart contract 1060.

The validation of the environmental data according to a smart contract comprises the formal check of the data relating to the source of the data, if it comes from an authorized device, and a check of the functioning of the detection process or the sensor. In one embodiment the formal check comprises recording the absence of measured environmental data in a predetermined time window whereby lack of data then replaces received environmental data. A second check is based on further smart contract conditions, usually in a second client oriented smart contract, wherein conditions provided by the client are checked as temperature measurement or acceleration measurements always below a specific threshold.

Any digital computer system or platform (exemplified herein as system 1000) can be configured or otherwise programmed to implement a method disclosed herein, and to the extent that a particular digital computer system is configured to implement such a method, it is within the scope and spirit of the disclosure. Once a digital computer system is programmed to perform particular functions pursuant to computer readable and executable instructions from program software that implements a method disclosed herein, it in effect becomes a special purpose computer particular to an embodiment of the method disclosed herein. The techniques necessary to achieve this are well known to those skilled in the art and thus are not further described herein. The methods and/or processes disclosed herein may be implemented as a computer program product such as, for example, a computer program tangibly embodied in an information carrier, for example, in a non-transitory tangible computer-readable or non-transitory tangible machine-readable storage device and/or in a transitory, propagated signal, for execution by or to control the operation of, a data processing apparatus including, for example, one or more programmable processors and/or one or more computers.

The terms “non-transitory computer-readable storage device” and “non-transitory machine-readable storage device” encompasses distribution media, intermediate storage media, execution memory of a computer, and any other medium or device capable of storing for later reading by a computer program implementing embodiments of a method disclosed herein. A computer program product can be deployed to be executed on one computer or on multiple computers at one site or distributed across multiple sites and interconnected by one or more communication networks.

A propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or using an instruction execution system, apparatus, or device.

Computer readable and executable instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable and executable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable and executable instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

In the discussion, unless otherwise stated, adjectives such as “substantially” and “about” that modify a condition or relationship characteristic of a feature or features of an embodiment of the invention, are to be understood to mean that the condition or characteristic is defined to within tolerances that are acceptable for operation of the embodiment for an application for which it is intended.

“Coupled with” means indirectly or directly “coupled with”.

It is important to note that the method may include is not limited to those diagrams or to the corresponding descriptions. For example, the method may include additional or even fewer processes or operations in comparison to what is described herein. In addition, embodiments of the method are not necessarily limited to the chronological order as illustrated and described herein.

It should be noted that where an embodiment refers to a condition of “above a threshold”, this should not be construed as excluding an embodiment referring to a condition of “equal or above a threshold”. Analogously, where an embodiment refers to a condition “below a threshold”, this should not to be construed as excluding an embodiment referring to a condition “equal or below a threshold”. A condition should be interpreted as being fulfilled if the value of a given parameter is above a threshold, then the same condition is considered as not being fulfilled if the value of the given parameter is equal or below the given threshold. Conversely, should a condition be interpreted as being fulfilled if the value of a given parameter is equal or above a threshold, then the same condition is considered as not being fulfilled if the value of the given parameter is below (and only below) the given threshold.

Terms used in the singular shall also include the plural, except where expressly otherwise stated or where the context otherwise requires.

In the description and claims of the present application, each of the verbs, “comprise” “include” and “have”, and conjugates thereof, are used to indicate that the object or objects of the verb are not necessarily a complete listing of components, elements or parts of the subject or subjects of the verb.

Unless otherwise stated, the use of the expression “and/or” between the last two members of a list of options for selection indicates that a selection of one or more of the listed options is appropriate and may be made.

Discussions herein utilizing terms such as, for example, “processing”, “computing”, “calculating”, “determining”, “establishing”, “analyzing”, “checking”, “estimating”, “deriving”, “selecting”, “inferring” “updating”, “recording” and/or the like, may refer to operation(s) and/or process(es) of a computer, a computing platform, a computing system, or other electronic computing device, that manipulate and/or transform data represented as physical (e.g., electronic) quantities within the computer's registers and/or memories into other data similarly represented as physical quantities within the computer's registers and/or memories or other information storage medium that may store instructions to perform operations and/or processes. The term “determining” and “estimating” may also refer to “heuristically determining” and “heuristically estimating”, respectively.

The term “operatively coupled” may encompass the meanings of the terms “responsively coupled”, “communicably coupled”, and the like.

It is appreciated that certain features of the invention, which are, for clarity, described in the context of separate embodiments or example, may also be provided in combination in a single embodiment. Conversely, various features of the invention, which are, for brevity, described in the context of a single embodiment, example and/or option, may also be provided separately or in any suitable sub-combination or as suitable in any other described embodiment, example or option of the invention. Certain features described in the context of various embodiments, examples and/or options are not to be considered essential features of those embodiments, unless the embodiment, example and/or option is inoperative without those elements.

It is noted that the term “exemplary” is used herein to refer to examples of embodiments and/or implementations, and is not meant to necessarily convey a more desirable use-case.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

It should be understood that where the claims or specification refer to “a” or “an” element and/or feature, such reference is not to be construed as there being only one of that element. Hence, reference to “an element” or “at least one element” for instance may also encompass “one or more elements”.

As used herein, unless otherwise specified, the use of the ordinal adjectives “first”, “second”, etc., to describe like objects, merely indicate that different instances of like objects are being referred to, and are not intended to imply that the objects so described must be in a given sequence, temporally, in ranking, and/or in any other manner.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

While the invention has been described with respect to a limited number of embodiments, these should not be construed as limitations on the scope of the invention, but rather as exemplifications of some of the embodiments.

LIST OF REFERENCE SIGNS

| | |
|---|---|
| 1000 | system |
| 1050 | ECM engine |
| 1060 | smart contract |
| 1200 | storage facility |
| 1200A, B | storage facility |
| 1210 | sensor |
| 1210A, B | sensor |
| 1213 | sensor processor |
| 1214 | sensor memory |
| 1217 | sensor communication module |
| 1218 | sensor user interface |
| 1219 | sensor power module |
| 1220 | perishable good |
| 1300 | computing device (ECM register) |
| 1300A, B, C | computing device |
| 1305 | ledger |
| 1305A, B, C | ledger |
| 1310 | device processor |
| 1320 | device memory |
| 1330 | device communication module |
| 1350 | device user interface |
| 1360 | device power module |
| 1400 | communication network |
| 3100 | measuring step of a physical quantity |
| 3200 | receiving data step of data descriptive of physical quantity |
| 3300 | checking validity step |
| 3400 | storing transaction data |
| 3500 | comparison step |

1. A method for verification of environmental data relating to conditions under which perishable goods are stored and/or transported, the method comprising:
measuring, by one or more sensors employed by one or more storage facilities, environmental data descriptive of a physical quantity that relates to environmental conditions to which the perishable goods are subjected to;
receiving, at a plurality of computing devices, said environmental data descriptive of the measured physical quantity;
checking validity of the received environmental data by at least one of the plurality computing devices to determine if the conditions relating to a smart contract comprising measurement conditions for the environmental data are met;
creating transaction data relating to the received environmental data based on the validity check;
transmitting said transaction data as well as the received environmental data to a plurality of distributed public ledgers, if the received environmental data is found to be valid; and
to ensure data integrity, storing said transaction data relating to the received environmental data in said plurality of distributed public ledgers, if the received environmental data is found to be valid,
wherein the plurality of distributed public ledgers are part of a blockchain system and the transaction data are stored in a blockchain.

2. The method of claim 1. wherein the step of measuring occurs along a transportation route of the perishable items, while the perishable items are transported.

3. The method of claim 1, wherein the one or more sensors include any one of the following:
inertial sensors or non-inertial sensors, wherein the non-inertial sensors comprise any one of the following: temperature sensors; humidity sensors; pressure sensors; light sensors; non-inertial sensors; gas sensors, air flow sensors; motion sensors, acceleration sensory, global positioning system sensors, or NOx sensors.

4. The method of claim 1, wherein the one or more sensors are employed to sense any one of the following physical quantities: temperature, pressure, relative humidity, or intensity of light.

5. The method according to claim 1, wherein the one or more sensors are associated with a secret key each to digitally sign environmental data provided by said sensor with an associated private key for identification purposes with a publically available public key.

6. The method according to claim 5, wherein the public key of each of the one or more sensors is also associated with reputation data descriptive of the reputation of said sensor, wherein subsequent validated sensor data increases the reputation value, whereas failure decreases the reputation value.

7. The method according to claim 5, wherein, to create an incentive to provide accurate environmental data, each sensor receives a tradable digital asset that includes micropayments in form of a digital currency, and wherein the incentive is proportional to the reputation of the sensor.

8. The method according to claim 1, wherein the smart contract within the step of checking the validity of the received environmental data comprises a formal check of the data relating to the source of the data, if it comes from an authorized device, and a check of the functioning of the detection process or the one or more sensors, wherein, in case of a non-functioning sensor, the formal check comprises recording an absence of measured environmental data in a predetermined time window.

9. The method according to claim 8, wherein after the step of checking the validity of the received environmental data, another check is based on further smart contract conditions, wherein one or more conditions related to at least one of a threshold or a range, of the received environmental data descriptive of a physical quantity is checked to determine if the conditions relating to the further smart contract comprising at least one of threshold conditions and range conditions for the environmental data are met and creating further transaction data, when a threshold or range transgressing value is detected when the initial validation check was validated.

10. A system for verification of environmental data relating to the conditions under which perishable goods are stored and/or transported, the system comprising:
one or more sensors for measuring environmental data descriptive of a physical quantity that relates to an environmental condition in and/or outside a storage facility used for storing and/or transporting perishable goods;
a plurality of computing devices that are operative and configured to receive said environmental data descriptive of the measured physical quantity,
wherein at least one of the plurality of computing devices is operative and configured to check validity of the received environmental data to determine if conditions relating to a smart contract comprising measurement conditions for the environmental data are met;
wherein said one of the plurality of computing devices having performed the validity check is configured to create transaction data relating to the received environmental data based on the validity check, is further configured to transmit said transaction data as well as the received environmental data to at least two of the plurality of distributed public ledgers, if the received environmental data is found to be valid;
wherein at least said two of the plurality of computing devices are operative to store said transaction data relating to the received environmental data in a respective at least two distributed public ledgers, if the received environmental data is found to be valid, and
wherein the plurality of distributed public ledgers are part of a blockchain system and the transaction data are stored in a blockchain.

11. The system of claim 10, wherein the one or more sensors are mounted directly on the packaging of perishable goods.

12. The system of claim 10, wherein the one or more sensors include any one of the following:
inertial sensors or non-inertial sensors, wherein the non-inertial sensors comprise at least one of the following: temperature sensors; humidity sensors; pressure sensors; light sensors; non-inertial sensors; gas sensors, air flow sensors; motion sensors; acceleration sensory; global positioning system sensors, or NOx sensors.

13. The system of claim 10, wherein the one or more sensors are operative to sense any one of the following physical quantities: temperature, pressure, relative humidity, or intensity of light.

14. The system according to claim 10, wherein a plurality of sensors is provided and each of the sensors comprise one of said computing devices and function as nodes of a blockchain and which is configured to be employed for validating and storing data records in respective ledgers.

15. The system according to claim 10, wherein a plurality of computing devices comprise at least one of said sensors.

16. The system according to claim 10, wherein, while encompassing positioning data in the set of environmental data and nevertheless reducing sensor power consumption, sensors, being free of any positioning units, are associated with positioning sensors of an associated storage facility.

17. (canceled)

18. A transitory or non-transitory computer readable storage medium storing a set of instructions that are executable by at least one processor of a server to cause the server to perform a method for verification of environmental data relating to the conditions under which perishable goods are stored and/or transported, the method comprising:
measuring, by one or more sensors mounted on and/or comprised in one or more storage facilities, environmental data descriptive of a physical quantity that relates to an environmental condition in a storage compartment of the storage facilities;
receiving, at a plurality of computing devices, environmental data descriptive of the measured physical quantity;
checking validity of the received environmental data by at least one of the computing devices to determine if conditions relating to a smart contract comprising measurement conditions for the environmental data are met;
creating transaction data relating to the received environmental data based on the validity check;
transmitting said transaction data as well as the received environmental data to a plurality of distributed public ledgers, if the received environmental data is found to be valid; and
to ensure data integrity, storing transaction data relating to the received environmental data in said plurality of distributed public ledgers, if the received environmental data is found to be valid,
wherein the plurality of distributed public ledgers are part of a blockchain system and the transaction data are stored in a blockchain.
